SEQUENCE LISTING (4 sequences)

SEQ ID NO: 1 - DNA, 24 bp, Dengue virus
  gene (1)..(24)
aaagaccagagatcctgctgtctc

SEQ ID NO: 2 - DNA, 25 bp, Dengue virus
  gene (1)..(25)
acgctgggagagaccagagatcctg

SEQ ID NO: 3 - DNA, 18 bp, Dengue virus
  primer_bind (1)..(18)
gttagaggagaccccccg

SEQ ID NO: 4 - DNA, 19 bp, Dengue virus
  primer_bind (1)..(19)
gcgttctgtgcctggaatg

(19) United States
(12) Patent Application Publication
(10) Pub. No.: US 2013/0130235 A1
(43) Pub. Date: May 23, 2013

(54) PROBES AND PRIMERS FOR DETECTION OF DENGUE

(71) Applicants: Manjula Jagannath, Bangalore (IN); Manoj Mulakkapurath Narayanan, Bangalore (IN); Chandrasekhar Bhaskaran Nair, Bangalore (IN); Pillarisetti Venkata Subbarao, Bangalore (IN)

(72) Inventors: Manjula Jagannath, Bangalore (IN); Manoj Mulakkapurath Narayanan, Bangalore (IN); Chandrasekhar Bhaskaran Nair, Bangalore (IN); Pillarisetti Venkata Subbarao, Bangalore (IN)

(73) Assignee: BIGTEC PRIVATE LIMITED, Banagalore (IN)

(21) Appl. No.: 13/812,863

(22) PCT Filed: Jul. 15, 2011
(86) PCT No.: PCT/IB11/53164

§ 371 Date: Jan. 28, 2013

(30) Foreign Application Priority Data
Jul. 29, 2010 (IN) 2162/CHE/2010

(51) Int. Cl. C12Q 1/70

(52) U.S. Cl. CPC C12Q 1/701
USPC 435/5; 536/24.32; 536/24.33

(57) ABSTRACT

The present disclosure gives description of a method used for the detection and quantification of dengue viral infection caused by dengue virus using nucleic acids isolated from blood, plasma or serum samples by employing Oligonucleotide probes. The method employed here for detection is by Real time PCR. The instant disclosure also provides for primers, probes, PCR Reaction mixture and kit thereof.

### TECHNICAL FIELD

The present disclosure is in relation to a method for the detection and quantification of Dengue virus from blood, plasma or serum samples by employing “Oligonucleotide” probes.

### BACKGROUND OF THE DISCLOSURE

The incidence of dengue has grown dramatically around the world in recent decades. Some 2.5 billion people—two fifths of the world's population—are now at risk from dengue. WHO currently estimates there may be 50 million dengue infections worldwide every year. In 2007 alone, there were more than 890,000 reported cases of dengue in the Americas, of which 26 000 cases were DHF. The disease is now endemic in more than 100 countries in Africa, the Americas, the Eastern Mediterranean, South-east Asia and the Western Pacific. South-east Asia and the Western Pacific are the most seriously affected. Before 1970 only nine countries had experienced DHF epidemics, a number that had increased more than four-fold by 1995. Dengue is a mosquito-borne infection that in recent decades has become a major international public health concern. Dengue is found in tropical and sub-tropical regions around the world, predominantly in urban and semi-urban areas. Dengue haemorrhagic fever (DHF), a potentially lethal complication, was first recognized in the 1950s during dengue epidemics in the Philippines and Thailand. Today DHF affects most Asian countries and has become a leading cause of hospitalization and death among children in the region. DENV is an ssRNA positive-strand virus of the family Flaviviridae, genus Flavivirus. It is also known as breakbone fever. There are four distinct, but closely related, viruses that cause dengue. Dengue haemorrhagic fever (DHF) is a potentially deadly complication that is characterized by high fever, often with enlargement of the liver, and in severe cases circulatory failure. The illness often begins with a sudden rise in temperature accompanied by facial flush and other flu-like symptoms. The fever usually continues for two to seven days and can be as high as 41° C., possibly with convulsions and other complications. There is no specific treatment for dengue fever. Currently used methods for the diagnosis of dengue is based on the serological detection of anti-dengue IgM and IgG in the serum by ELISA. These serological methods are unable to detect the infection during the early phase of the disease. Thus there is a need for rapid and sensitive methods for detection of dengue infection early in the course of infection for better patient management.

### STATEMENT OF THE DISCLOSURE

Accordingly, the present disclosure relates to a probe having nucleotide sequence set forth as SEQ ID Nos. 1 or 2, optionally conjugated with detectable labels; primer having nucleotide sequence set forth as SEQ ID Nos. 3 or 4; a PCR reaction mixture for detection of dengue infection, said mixture comprising nucleic acid amplification reagents, probe having nucleotide sequence selected from a group comprising SEQ ID Nos. 1 and 2 or a combination of probes thereof, and primers having nucleotide sequence set forth as SEQ ID Nos. 3 and 4; a method of detecting and optionally quantifying dengue infection, said method comprising acts of—(a) obtaining a PCR reaction mixture comprising nucleic acid amplification reagents, probe selected from a group comprising SEQ ID No.1 and 2 and primers having nucleotide sequence set forth as SEQ ID Nos. 3 and 4, (b) introducing test sample to the PCR reaction mixture for PCR amplification to obtain copies of target sequence, followed by measuring fluorescence signal generated for detecting the dengue infection and (c) optionally, constructing a Standard Curve from the detected signal for quantifying the dengue infection; a kit for detecting dengue infection, said kit comprising probe having nucleotide sequence selected from a group comprising SEQ ID Nos. 1 and 2 or a combination of probes thereof, optionally labeled at 5′ and 3′ end, primers having nucleotide sequence set forth as SEQ ID Nos. 3 and 4, and amplification reagents, optionally along with instruction manual; and a method of assembling a kit for detection of dengue infection, said method comprising step of combining probe having nucleotide sequence selected from a group comprising SEQ ID Nos. 1 and 2 or a combination of probes thereof, primers having nucleotide sequence set forth as SEQ ID Nos. 3 and 4, and amplification reagents, optionally along with instruction manual.

### BRIEF DESCRIPTION OF ACCOMPANYING FIGURES

In order that the disclosure may be readily understood and put into practical effect, reference will now be made to exemplary embodiments as illustrated with reference to the accompanying figures. The figure together with a detailed description below, are incorporated in and form part of the specification, and serve to further illustrate the embodiments and explain various principles and advantages, in accordance with the present disclosure where:

FIG. 1 shows amplification plot for dengue serotypes by real time PCR using SEQ ID No. 1.

FIG. 2 shows amplification plot for dengue serotypes by real time PCR using SEQ ID No.2.

FIG. 3 shows amplification plot for dengue serotypes by real time PCR using National reference laboratory primers & probe.

FIG. 4 shows a log dilution curve.

### DETAILED DESCRIPTION OF THE DISCLOSURE

The present disclosure relates to probe having nucleotide sequence set forth as SEQ ID Nos. 1 or 2, optionally conjugated with detectable labels.

In an embodiment of the present disclosure, the probe is for detecting dengue infection; and wherein the detectable labels are flurophore at 5′ end and quencher at 3′ end.
In another embodiment of the present disclosure, the fluorophore is selected from a group comprising fluorescein, fluorescein derivatives consisting of 6-Carboxy Fluorescein [FAM], VIC, JOE, 5-(2′-aminoethyl)aminonaphthalene-1-sulphonic acid, coumarin and coumarin derivatives, lucifer yellow, texas red, tetramethylrhodamine, tetrachloro-6-carboxyfluoroscein, 5-carboxyrhodamine and cyanine dyes, preferably 6-Carboxy Fluorescein [FAM]; and the quencher is selected from a group comprising tetra methyl rhodamine, 4′-(4-dimethylaminophenylazo)benzoic acid, 4-dimethylaminophenyl azophenyl-4′-maleimide, tetramethylrhodamine, carboxytetramethyl rhodamine and black hole quencher 1 [BHQ] dyes, preferably black hole quencher 1 (BHQ1).
The present disclosure relates to primer having nucleotide sequence set forth as SEQ ID Nos. 3 or 4.
In an embodiment of the present disclosure, the primer having the SEQ ID No. 3 is sense primer and the primer having the SEQ ID No. 4 is an antisense primer.
In another embodiment of the present disclosure, the primers correspond to probe having SEQ ID Nos. 1 or 2; and wherein the primers are used in combination with either the probe having SEQ ID No. 1 or SEQ ID No. 2.
The present disclosure relates to a PCR reaction mixture for detection of dengue infection, said mixture comprising nucleic acid amplification reagents, probe having nucleotide sequence selected from a group comprising SEQ ID Nos. 1 and 2 or a combination of probes thereof; and primers having nucleotide sequence set forth as SEQ ID Nos. 3 and 4.
In an embodiment of the present disclosure, the primer having the SEQ ID No. 3 is sense primer and the primer having the SEQ ID No. 4 is antisense primer; and the probe is conjugated with detectable labels having flurophore at 5′ end and quencher at 3′ end.
In another embodiment of the present disclosure, the primers correspond to probe having SEQ ID Nos. 1 or 2; and wherein the primers are used in combination with either the probe having SEQ ID No. 1 or SEQ ID No. 2.
In yet another embodiment of the present disclosure, the dengue infection is detected from sample selected from a group comprising blood, plasma and serum or any combination thereof; and the amplification reagents are selected from a group comprising magnesium chloride, Taq polymerase and buffer or any combination thereof.

The present disclosure relates to a method of detecting and optionally quantifying dengue infection, said method comprising acts of:
(a) obtaining a PCR reaction mixture comprising nucleic acid amplification reagents, probe selected from a group comprising SEQ ID No. 1 and 2 and primers having nucleotide sequence set forth as SEQ ID Nos. 3 and 4;
(b) introducing test sample to the PCR reaction mixture for PCR amplification to obtain copies of target sequence, followed by measuring fluorescence signal generated for detecting the dengue infection; and
(c) optionally, constructing a Standard Curve from the detected signal for quantifying the dengue infection.
   In an embodiment of the present disclosure, the primer having the SEQ ID No. 3 is sense primer and the primer having the SEQ ID No. 4 is an antisense primer.
   In another embodiment of the present disclosure, the primers correspond to probe having SEQ ID Nos. 1 or 2; and wherein the primers are used in combination with either the probe having SEQ ID No. 1 or SEQ ID No. 2.
   In yet another embodiment of the present disclosure, the test sample is selected from a group comprising blood, plasma and serum or any combination thereof; and the amplification reagents are selected from a group comprising magnesium chloride, Taq polymerase and buffer or any combination thereof.
   In still another embodiment of the present disclosure, the probe is conjugated with detectable labels having flurophore at 5′ end and quencher at 3′ end; and the fluorescence signal is generated by the probes having flurophore at 5′ end and quencher at 3′ end.
   In still another embodiment of the present disclosure, the fluorophore is selected from a group comprising fluorescein, fluorescein derivatives consisting of 6-Carboxy Fluorescein [FAM], VIC, JOE, 5-(2′-aminoethyl)aminonaphthalene-1-sulphonic acid, coumarin and coumarin derivatives, lucifer yellow, texas red, tetramethylrhodamine, tetrachloro-6-carboxyfluoroscein, 5-carboxyrhodamine and cyanine dyes, preferably 6-Carboxy Fluorescein [FAM]; and the quencher is selected from a group comprising tetra methyl rhodamine, 4′-(4-dimethylaminophenylazo)benzoic acid, 4-dimethylaminophenyl azophenyl-4′-maleimide, tetramethylrhodamine, carboxytetramethyl rhodamine and black hole quencher 1 [BHQ] dyes, preferably black hole quencher 1 (BHQ1).
   The present disclosure relates to a kit for detecting dengue infection, said kit comprising probe having nucleotide sequence selected from a group comprising SEQ ID Nos. 1 and 2 or a combination of probes thereof, optionally labeled at 5′ and 3′ end; primers having nucleotide sequence set forth as SEQ ID Nos. 3 and 4; and amplification reagents, optionally along with instruction manual.
   In another embodiment of the present disclosure, the probe is conjugated with detectable labels having flurophore at 5′ end and quencher at 3′ end; and the amplification reagents are selected from a group comprising magnesium chloride, Taq polymerase and buffer or any combination thereof.
   The present disclosure relates to a method of assembling a kit for detection of dengue infection, said method comprising step of combining probe having nucleotide sequence selected from a group comprising SEQ ID Nos. 1 and 2 or a combination of probes thereof; primers having nucleotide sequence set forth as SEQ ID Nos. 3 and 4; and amplification reagents, optionally along with instruction manual.

### Regions Chosen For Primers and Probes Designing

In an embodiment of the present disclosure, primers and probes are designed for a region which is conserved in all the four serotypes. Probes having SEQ ID No. 1 or SEQ ID No.2 along with primers having SEQ ID No. 3 and SEQ ID No. 4 can detect all the four serotypes of dengue virus.

The objective of the present disclosure is detection of dengue viral infection caused by dengue virus from RNA isolated from infected blood, serum or plasma of an infected person. The mode of detection is by monitoring increase in fluorescence by real time PCR using “Oligonucleotide” probes labeled with fluorophore and quencher.

The present disclosure is with regard to the detection of dengue viral infection using Oligonucleotide probes and their respective primers employing real time PCR method. The above mentioned “Oligonucleotide” probes are conjugated to a fluorophore at the 5′ end and a quencher at the 3′ end. The fluorophore used in the current invention is FAM (6-Carboxy Fluorescein). Apart from 6-carboxy fluorescein, other fluorophores selected from the group comprising fluorescein and fluorescein derivatives FAM, VIC, JOE, 5-(2′-aminoethyl)aminonaphthalene-1-sulphonic acid, coumarin and coumarin derivatives, lucifer yellow, texas red, tetramethylrhodamine, tetrachloro-6-carboxyfluoroscein, 5-carboxyrhodamine and cyanine dyes can also be used for labelling.

The quencher used in the current invention is BHQ1 (Black hole quencher 1). Apart from BHQ1 other quenchers selected from the group comprising Tetra Methyl Rhodamine, 4′-(4-dimethylaminophenylazo) benzoic acid, 4-dimethylaminophenyl azophenyl-4′-maleimide, tetramethylrhodamine, carboxytetramethylrhodamine and BHQ dyes can also be used for labelling.

In another embodiment of the present disclosure said fluorophore is 6-Carboxy Fluorescein [FAM] and the quencher is Black hole quencher 1 [BHQ1] when present at the 3′ end.

According to the present disclosure the probes designated by SEQ ID No. 1 or SEQ ID No. 2 along with primers designated as SEQ ID No. 3 & SEQ ID No. 4 are designed for the detection of dengue viral infection The present disclosure is in relation to a method for detecting dengue viral infection, where in the said PCR mixture comprising of nucleic acid amplification reagents, oligonucleotide probes designated as SEQ ID No.1 or SEQ ID No. 2 along with their corresponding primers and dengue RNA sample is subjected for amplification using real-time PCR to obtain copies of the target sequence. The amplification is measured in terms of increase in fluorescence signal and the amount of signal produced is compared with uninfected samples.

The detection of the dengue infection is followed by the optional construction of a Standard Curve from the detected signal to obtain copy number for quantifying dengue infection.

According to the present disclosure oligonucleotide probes are having a size ranging from 24-25 nucleotides. The designed probes have a fluorophore at the 5′ end and quencher at the 3′ end.

The fluorophore at the 5′ end is FAM (6-Carboxy Fluorescein) and the quencher is Black hole quencher 1 [BHQ1] when present at the 3′ end.

The current disclosure is used for the detection of dengue viral infection caused by dengue virus using RNA isolated from blood, serum or plasma samples. The method employed for detection is by using real time PCR.

According to the present disclosure the “Oligonucleotide probe” refers to a short sequence of deoxyribonucleic acid (DNA). The Oligonucleotide probe can specifically hybridise to the target DNA without exhibiting non-specific hydridisation to uninfected DNA.

The probes employed here follow the principles of Taqman chemistry. TaqMan probes also called Double-Dye oligonucleotide or dual labeled probes, are the most widely used type of probes.

The oligonucleotide probes according to the present disclosure is further provided with respective sense and anti-sense primers that can be used to specifically amplify and detect dengue viral infections caused by dengue virus by real time PCR. The primers as claimed above have a size ranging from 18-19 nucleotides. The corresponding probes and primer sequences for the detection of dengue viral infection is as shown in table 1.

TABLE 1

| Sequence | |
|---|---|
| name | Nucleotide sequence |
| SEQ ID | 5′-FAM-AAAGACCAGAGATCCTGCTGTCTC-BHQ1-3′ |
| No. 1 | Or |
| | 5′-Flurophore-AAAGACCAGAGATCCTGCTGTCTC- |
| | Quencher-3′ |
| SEQ ID | 5′-FAM-ACGCTGGGAGAGACCAGAGATCCTG-BHQ1-3′ |
| No. 2 | Or |
| | 5′-Flurophore-ACGCTGGGAGAGACCAGAGATCCTG- |
| | Quencher-3′ |
| SEQ ID | 5′-GTTAGAGGAGACCCCCCG-3′ |
| No. 3 | |
| SEQ ID | 5′-GCGTTCTGTGCCTGGAATG-3′ |
| No. 4 | |

The primers and probes disclosed in the current invention are also be provided in the form of a kit along with an instruction manual. The kit contains PCR amplification reagents such as dNTPs, Taq DNA polymerase, magnesium chloride etc along with the disclosed primers and probes. The oligonucleotide probes according to present disclosure find application for the detection of dengue viral infection caused by dengue virus.

The efficiency of these probes and primers in detecting dengue viral infection is illustrated by the following examples. The present disclosure is further elaborated by the following examples and figures. However, these examples should not be construed to limit the scope of the disclosure.

### EXAMPLE 1

RNA is isolated from cultures of all the four types of dengue viruses (dengue virus type 1, dengue virus type 2, dengue virus type 3 & dengue virus type 4) using a commercial RNA isolation kit. The purified RNA is subjected to Real time PCR using probes of either SEQ ID No. 1 or SEQ ID No. 2 along with primers of SEQ ID No. 3 and 4, Similarly the RNA from all the four serotypes are tested with primers and probe designed by a National reference laboratory for the detection of dengue viral infection. Same concentrations of Real time-PCR reagents, template and primers are used in each case and also cycling conditions are kept constant for all the reactions. The composition of PCR mix and PCR conditions are as given in Table 2 & Table 3.

TABLE 2
Real time-PCR mix composition
Real time PCR mix Composition

| | |
|---|---|
| Superscript III reverse transcriptase | 0.2 μl |
| and platinum taq DNA polymerase mix | |
| Commercial Premix | 5.0 μl |
| Forward Primer | 0.3 μl (5 picomoles) |
| Reverse Primer | 0.3 μl (5 picomoles) |
| Probe | 0.2 μl (2 picomoles) |
| Sample | 4.0 μl |
| Total volume | 10 μl |

TABLE 3
Real time-PCR cycle conditions
PCR Program

| | |
|---|---|
| Step 1 (cDNA synthesis) | 50° C. for 10 min |
| Step 2 (initial denaturation) | 95° C. for 120 sec |
| Step 3 (cycle denaturation) | 95° C. for 15 sec |
| Step 4 (annealing & extension) | 60° C. for 34 sec |

### Step 2 and 3 are Repeated 45 Times

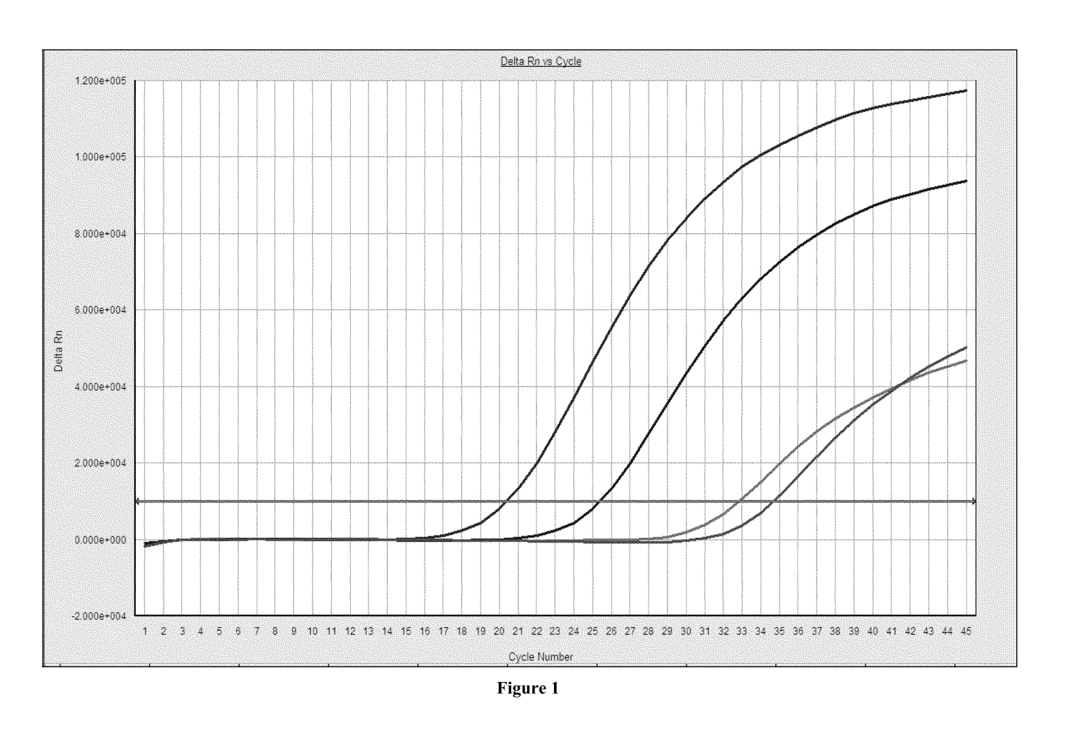

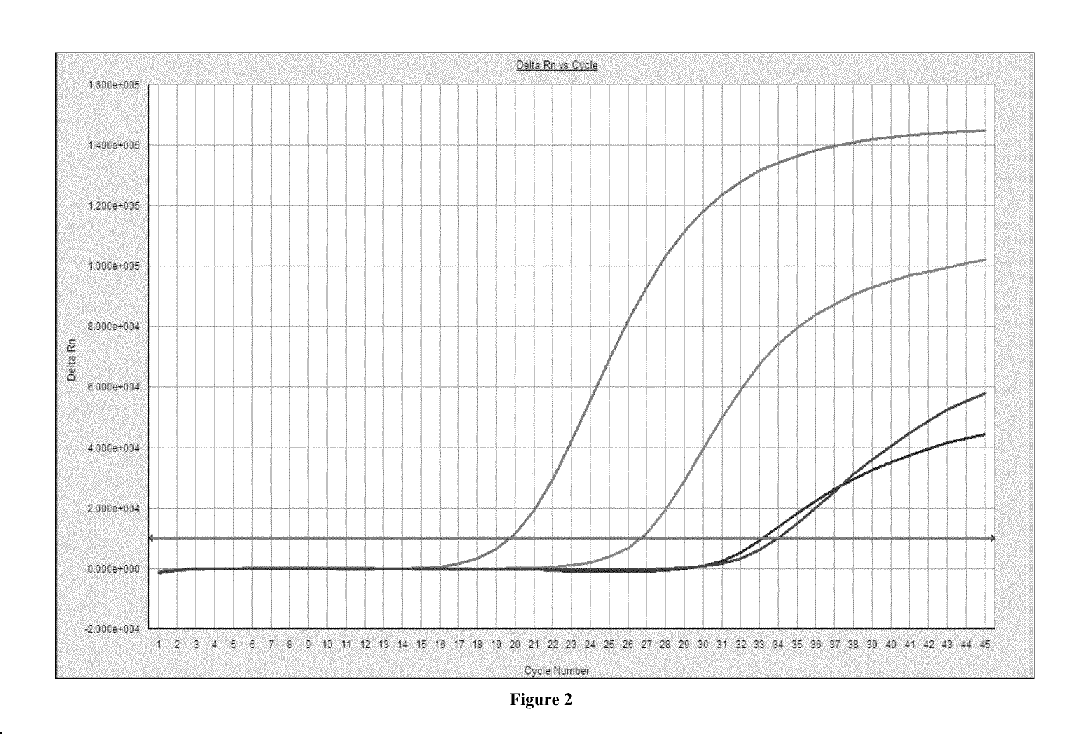

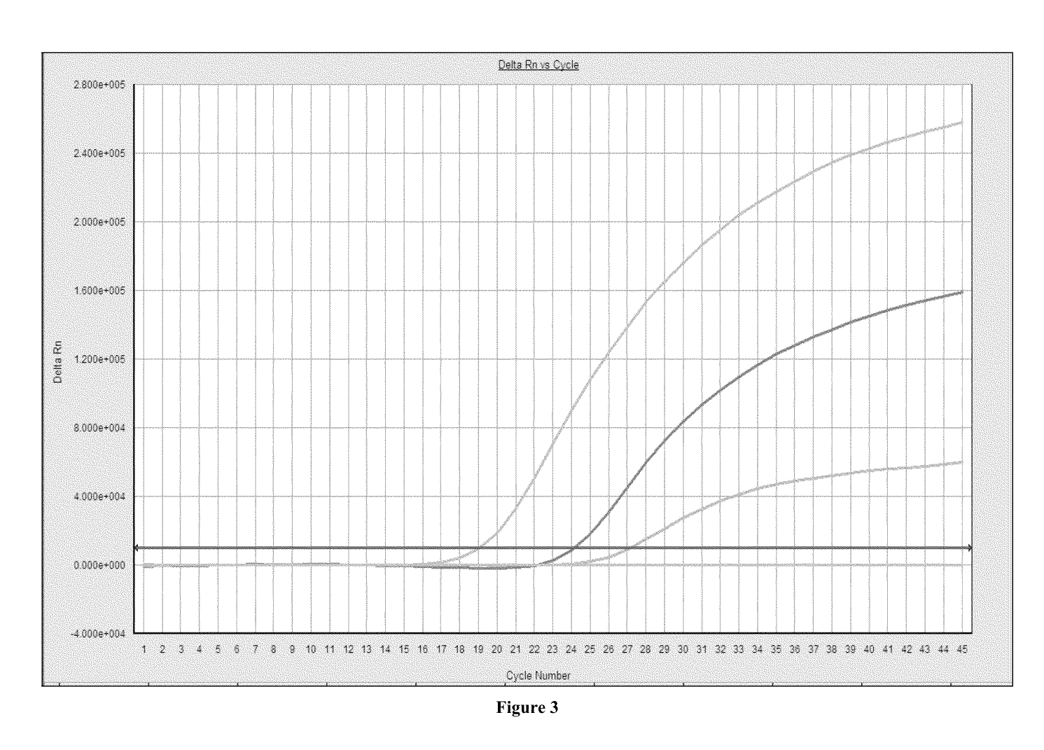

Results obtained showed that the probes designated as SEQ ID No. 1 and SEQ ID No. 2 detected all the four types of dengue viruses within 45 cycles (positive sample cutoff) showing 100% specificity and sensitivity (Table. 4, FIG. 1, FIG. 2 & FIG. 3). The National reference laboratory probe and primers detected only the three dengue viruses namely dengue virus type 1, dengue virus type 2, dengue virus type 3 and it failed to detect dengue virus type 4.

TABLE 4

| | | | Ct National |
|---|---|---|---|
| | Ct SEQ | Ct SEQ | reference |
| Sample ID | ID No 1 | ID No 2 | laboratory |
| Dengue virus type 1 | 32.9 | 33.1 | 27.15 |
| Dengue virus type 2 | 25.4 | 26.6 | 24.16 |
| Dengue virus type 3 | 20.4 | 19.68 | 19 |
| Dengue virus type 4 | 34.7 | 34 | Undetected |

### EXAMPLE 2

RNA is isolated from 25 clinical serum samples using a commercial RNA isolation kit. The purified RNA is subjected to real time PCR using the probes designated as SEQ ID No. 1 or SEQ ID No. 2 along with primers of SEQ ID No. 3 and 4, as well as the primers and probes designed by National reference laboratory. Same concentrations of Real time-PCR reagents, template and primers are used in each case and also cycling conditions are kept constant for all the reactions.

Results with the clinical samples suggested that the probes designated as SEQ ID No. 1 and SEQ ID No.2 detected all the 25 clinical samples successfully, While the primers and probe from the National reference laboratory could detect only 14 out of 25 clinical samples (Table. 5). This data strongly supports the sensitivity of probes designated as SEQ ID No. 1 and SEQ ID No. 2 along with their respective primers in detecting dengue viral infections.

TABLE 5
Ct values by real time PCR

| Sample | Ct SEQ | Ct SEQ | Ct National |
|---|---|---|---|
| name | ID No 1 | ID No 2 | reference lab |
| Sample 1 | 30.9 | 31.4 | 33.1 |
| Sample 2 | 31.7 | 30.8 | 32.2 |
| Sample 3 | 31.3 | 31.9 | 34.2 |
| Sample 4 | 33.9 | 33.5 | 33.1 |
| Sample 5 | 25.3 | 25.9 | 26.1 |
| Sample 6 | 38.5 | 38.0 | Undetected |
| Sample 7 | 35.8 | 35.3 | Undetected |
| Sample 8 | 27.7 | 28.1 | 29.4 |
| Sample 9 | 30.3 | 30.8 | 29.6 |
| Sample 10 | 32.3 | 31.9 | 36.6 |
| Sample 11 | 31.8 | 32.3 | 32.1 |
| Sample 12 | 31.9 | 31.4 | 30.7 |
| Sample 13 | 38.7 | 37.9 | Undetected |
| Sample 14 | 35.5 | 35.1 | Undetected |
| Sample 15 | 34.5 | 35.0 | Undetected |
| Sample 16 | 18.95 | 19.1 | 20.6 |
| Sample 17 | 30.7 | 31.0 | 30.69 |
| Sample 18 | 36.1 | 35.8 | Undetected |
| Sample 19 | 34.2 | 33.8 | Undetected |
| Sample 20 | 35.63 | 35.2 | Undetected |
| Sample 21 | 32.0 | 32.4 | 31.61 |
| Sample 22 | 33.03 | 32.9 | Undetected |
| Sample 23 | 31.0 | 31.3 | 37.5 |
| Sample 24 | 31.4 | 31.7 | Undetected |
| Sample 25 | 35.1 | 34.8 | Undetected |

### EXAMPLE 3

RNA is isolated from 4 whole blood samples obtained from patients suffering from high fever as the routine laboratory tests (IgM & IgG serology tests) could not confirm the kind of infection in these cases. The purified RNA is subjected to real time PCR using the probes designated as SEQ ID No. 1 or SEQ ID No. 2 along with primers of SEQ ID No. 3 and 4. The results obtained showed that the probes designated as SEQ ID No. 1 and SEQ ID No. 2 could detect all the 4 clinical blood samples as positive for dengue infection (Table. 6). The Ct values obtained for these samples were very late indicating that the patients are in the early stage of infection and that is the reason for the failure of the routine laboratory tests.

TABLE 6
Ct values by real time PCR

| Sample | Ct SEQ | Ct SEQ |
|---|---|---|
| name | ID No 1 | ID No 2 |
| Patient 1 | 38.0 | 37.2 |
| Patient 2 | 36.5 | 36.9 |
| Patient 3 | 35.8 | 35.3 |
| Patient 4 | 37.4 | 37.1 |

### EXAMPLE 4

This study is done in order to show that even plasma samples are used for the detection of dengue infections. RNA is isolated from 5 clinical plasma samples using a commercial kit. The extracted RNA is subjected to real time PCR using probes designated as SEQ ID No. 1 or SEQ ID No. 2 along with primers of SEQ ID No. 3 and 4. The results obtained showed that the probes designated as SEQ ID No. 1 and SEQ ID No. 2 could detect all the 5 clinical plasma samples as positive for dengue infection (Table. 7).

TABLE 7
Ct values by real time PCR

| Sample | Ct SEQ | Ct SEQ |
|---|---|---|
| name | ID No 1 | ID No 2 |
| Sample 1 | 28.1 | 27.5 |
| Sample 2 | 30.3 | 30.9 |
| Sample 3 | 26.9 | 27.3 |
| Sample 4 | 36.5 | 36.4 |
| Sample 5 | 32.6 | 33.0 |

### EXAMPLE 5

One can also quantify the viral load from an infected sample by comparing the Ct values obtained from a standard curve.

### Protocol for Calculation of Viral Copy Number

Around 25 microlitre of PCR reaction mix containing dengue RNA is subjected to PCR along with the respective primers using a conventional PCR machine. After PCR the amplified samples are run on an agarose gel and stained with ethidium bromide. The amplicon band is then excised from the gel and purified using a Qiaquick gel extraction kit. The absorbance (2 μl amplicon) is estimated at 260 nm using a nanodrop. Extinction coefficient of the amplicon is calculated from individual base coefficient by summing up.

Nanomoles of amplicon is calculated using the following equation:

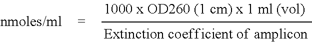

Copy number is calculated using the formula:

Copy number/ml=(Moles/ml)×Avogadro number.

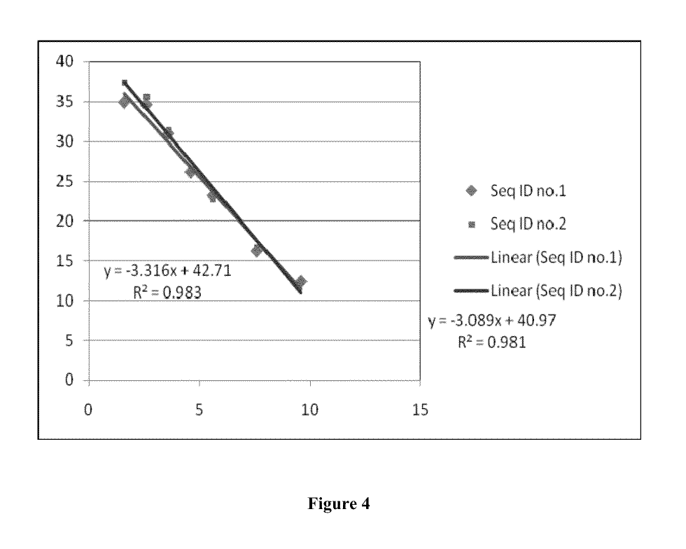

From the copy number of the pure amplicon a standard curve is generated by running 10¹² to 10³ dilutions of the amplicon using a real-time PCR. From the Ct obtained from the standard curve, viral copy number can be calculated for unknown samples (FIG. 4, & Table.8).

TABLE 8
log dilution curve values with respect to Ct

| Log10 | Dilution per 4 μl | DNA taken | Ct Seq | Ct Seq |
|---|---|---|---|---|
| dilution | taken for PCR | for PCR | ID no. 1 | ID no. 1 |
| 1.00E+12 | 3.90E+09 | 9.591065 | 12.36 | 12.37 |
| 1.00E+10 | 3.90E+07 | 7.591065 | 16.23 | 16.72 |
| 1.00E+08 | 3.90E+05 | 5.591065 | 23.16 | 22.68 |
| 1.00E+07 | 3.90E+04 | 4.591065 | 26.13 | 26.38 |
| 1.00E+06 | 3.90E+03 | 3.591065 | 30.95 | 31.37 |
| 1.00E+05 | 3.90E+02 | 2.591065 | 34.57 | 35.6 |
| 1.00E+04 | 3.90E+01 | 1.591065 | 34.86 | 37.38 |
| 1.00E+03 | 3.90E+00 | 0.591065 | 38.23 | 35.35 |

### CONCLUSION

1. The oligonucleotide probes, SEQ ID No.1, and SEQ ID No.2 detected all the four types of dengue viruses showing that they are 100% specific and 100% sensitive.

2. The National reference laboratory probe detected only three dengue viruses namely dengue virus type 1, dengue virus type 2, dengue virus type 3 and it failed to detect dengue virus type 4.

3. The oligonucleotide probes, SEQ ID No.1, and SEQ ID No.2 along with their respective primers are more sensitive in detecting the clinical samples as compared to the Probes and primers of national reference laboratory.

4. Finally, the probes, SEQ ID No.1, and 2 SEQ ID No.2 along with their respective primers can detect the cases of dengue viral infections in blood, plasma or serum samples effectively.

We claim:

1) Probe consisting of nucleotide sequence set forth as SEQ ID Nos. 1 or 2, optionally conjugated with detectable labels.

2) The probe as claimed in claim 1, wherein the probe is for detecting dengue infection; and wherein the detectable labels are flurophore at 5′ end and quencher at 3′ end.

3) The probe as claimed in claim 2, wherein the fluorophore is selected from a group comprising fluorescein, fluorescein derivatives consisting of 6-Carboxy Fluorescein [FAM], VIC, JOE, 5-(2′-aminoethyl)aminonaphthalene-1-sulphonic acid, coumarin and coumarin derivatives, lucifer yellow, texas red, tetramethylrhodamine, tetrachloro-6-carboxyfluoroscein, 5-carboxyrhodamine and cyanine dyes, preferably 6-Carboxy Fluorescein [FAM]; and the quencher is selected from a group comprising tetra methyl rhodamine, 4′-(4-dimethylaminophenylazo)benzoic acid, 4-dimethylaminophenyl azophenyl-4′-maleimide, tetramethylrhodamine, carboxytetramethyl rhodamine and black hole quencher 1 [BHQ] dyes, preferably black hole quencher 1 (BHQ1).

4) Primer consisting of nucleotide sequence set forth as SEQ ID Nos. 3 or 4.

5) The primer as claimed in claim 4, wherein the primer consisting of the SEQ ID No. 3 is sense primer and the primer consisting of the SEQ ID No. 4 is an antisense primer.

6) The primer as claimed in claim 4, wherein the primers correspond to probe consisting of SEQ ID Nos. 1 or 2; and wherein the primers are used in combination with either the probe consisting of SEQ ID No. 1 or SEQ ID No. 2.

7) A PCR reaction mixture for detection of dengue infection, said mixture comprising nucleic acid amplification reagents, probe consisting of nucleotide sequence selected from a group comprising SEQ ID Nos. 1 and 2 or a combination of probes thereof; and primers consisting of nucleotide sequence set forth as SEQ ID Nos. 3 and 4.

8) The reaction mixture as claimed in claim 7, wherein the primer consisting of the SEQ ID No. 3 is sense primer and the primer consisting of the SEQ ID No. 4 is antisense primer; and the probe is conjugated with detectable labels having flurophore at 5′ end and quencher at 3′ end.

9) The reaction mixture as claimed in claim 7, wherein the primers correspond to probe consisting of SEQ ID Nos. 1 or 2; and wherein the primers are used in combination with either the probe consisting of SEQ ID No. 1 or SEQ ID No. 2.

10) The reaction mixture as claimed in claim 7, wherein the dengue infection is detected from sample selected from a group comprising blood, plasma and serum or any combination thereof; and the amplification reagents are selected from a group comprising magnesium chloride, Taq polymerase and buffer or any combination thereof.

11) A method of detecting and optionally quantifying dengue infection, said method comprising acts of:
obtaining a PCR reaction mixture comprising nucleic acid amplification reagents, probe selected from a group comprising SEQ ID No.1 and 2 and primers consisting of nucleotide sequence set forth as SEQ ID Nos. 3 and 4;
introducing test sample to the PCR reaction mixture for PCR amplification to obtain copies of target sequence, followed by measuring fluorescence signal generated for detecting the dengue infection; and
optionally, constructing a Standard Curve from the detected signal for quantifying the dengue infection.

12) The method as claimed in claim 11, wherein the primer consisting of SEQ ID No. 3 is sense primer and the primer consisting of SEQ ID No. 4 is an antisense primer.

13) The method as claimed in claim 11, wherein the primers correspond to probe consisting of SEQ ID Nos. 1 or 2; and wherein the primers are used in combination with either the probe consisting of SEQ ID No. 1 or SEQ ID No. 2.

14) The method as claimed in claim 11, wherein the test sample is selected from a group comprising blood, plasma and serum or any combination thereof; and the amplification reagents are selected from a group comprising magnesium chloride, Taq polymerase and buffer or any combination thereof.

15) The method as claimed in claim 11, wherein the probe is conjugated with detectable labels having flurophore at 5′ end and quencher at 3′ end; and the fluorescence signal is generated by the probes having flurophore at 5′ end and quencher at 3′ end.

16) The method as claimed in claim 15, wherein the fluorophore is selected from a group comprising fluorescein, fluorescein derivatives consisting of 6-Carboxy Fluorescein [FAM], VIC, JOE, 5-(2′-aminoethyl)aminonaphthalene-1-sulphonic acid, coumarin and coumarin derivatives, lucifer yellow, texas red, tetramethylrhodamine, tetrachloro-6-carboxyfluoroscein, 5-carboxyrhodamine and cyanine dyes, preferably 6-Carboxy Fluorescein [FAM]; and the quencher is selected from a group comprising tetra methyl rhodamine, 4′-(4-dimethylaminophenylazo)benzoic acid, 4-dimethylaminophenyl azophenyl-4′-maleimide, tetramethylrhodamine, carboxytetramethyl rhodamine and black hole quencher 1 [BHQ] dyes, preferably black hole quencher 1 (BHQ1).

17) A kit for detecting dengue infection, said kit comprising probe having nucleotide sequence selected from a group comprising SEQ ID Nos. 1 and 2 or a combination of probes thereof, optionally labeled at 5′ and 3′ end; primers consisting of nucleotide sequence set forth as SEQ ID Nos. 3 and 4; and amplification reagents, optionally along with instruction manual.

18) The kit as claimed in claim 17, wherein the probe is conjugated with detectable labels having flurophore at 5′ end and quencher at 3′ end; and the amplification reagents are selected from a group comprising magnesium chloride, Taq polymerase and buffer or any combination thereof.

19) A method of assembling a kit for detection of dengue infection, said method comprising step of combining probe consisting of nucleotide sequence selected from a group comprising SEQ ID Nos. 1 and 2 or a combination of probes thereof; primers consisting of nucleotide sequence set forth as SEQ ID Nos. 3 and 4; and amplification reagents, optionally along with instruction manual.
